(19) United States
(12) Patent Application Publication
(10) Pub. No.: US 2024/0149070 A1
(43) Pub. Date: May 9, 2024

(54) ELECTRIC POTENTIAL GENERATION METHOD, ELECTRIC POTENTIAL GENERATION APPARATUS, AND PROGRAM

(71) Applicant: Toshihiko HOSOYA, Tokyo (JP)

(72) Inventor: Toshihiko HOSOYA, Tokyo (JP)

(21) Appl. No.: 18/387,072

(22) Filed: Nov. 6, 2023

(30) Foreign Application Priority Data
Nov. 7, 2022 (JP) 2022-178346

(51) Int. Cl. A61N 2/00; G01R 33/54

(52) U.S. Cl. CPC A61N 2/006; G01R 33/543; A61N 2/02

(57) ABSTRACT

An electric potential generation method that includes generating a magnetic field having an intensity based on an arbitrary first temporal pattern in a region including a target position inside an object, using a magnetic field formation unit that generates a magnetic field: generating one or more ultrasonic waves in which respective parameters of direction, frequency, amplitude, and phase are separately adjusted, by separately controlling one or more wave sources; generating a force having a desired direction, intensity, and shape in accordance with the first temporal pattern so as to move a prescribed region of the object at the target position based on the force, by irradiating the target position with the one or more ultrasonic waves; and generating an arbitrary electric potential based on the magnetic field in the prescribed region.

### CROSS REFERENCE TO RELATED APPLICATION

Priority is claimed on Japanese Patent Application No. 2022-178346, filed Nov. 7, 2022, the content of which is incorporated herein by reference.

### BACKGROUND OF THE INVENTION

### Field of the Invention

The present invention relates to an electric potential generation method, an electric potential generation apparatus, and a program.

### Description of Related Art

Methods for generating electric potentials, electric fields, or electric currents at arbitrary positions in space or in an article are being studied. If an electric potential or an electric current having a desired spatial pattern or temporal pattern could be generated inside an article non-destructively, various applications could be contemplated, such as non-invasively stimulating the brain. Methods for generating local electric potentials or electric currents inside articles are known. For example. Non-Patent Document 1 (Michael G. Christiansen, et al., “Magnetic Strategies for Nervous System Control”. Annu. Rev. Neurosci., 42: 271-293, 2019 Apr. 2) describes a method for generating an induced electric current by radiating an intense magnetic field that varies over time.

When applying brain stimulation inside the brain, etc., it is required to generate desired electric potentials at desired positions with desired time intervals. Magnetic fields have the characteristic that they fundamentally cannot be localized in space, and quickly disperse when becoming distant from the generation source. For this reason, with the method described in Non-Patent Document 1, it was difficult to focus the magnetic field, and the range affected by the magnetic field was limited to areas near the magnetic field generator. For this reason, when trying to stimulate the brain based on the method described in Non-Patent Document 1, the effects thereof were limited to the brain surface and the resolution was approximately on the order of centimeters, and there were cases in which an electric potential, an electric field or an electric current could not be generated at an arbitrary position.

Additionally, Non-Patent Document 2 (Nir Grossman, et al., “Noninvasive Deep Brain Stimulation via Temporally Interfering Electric Fields”, Cell, 169, 1029-1041, 2017) describes a method for generating localized electric field fluctuations by means of interference, by applying, to an article, an electric potential that fluctuates between multiple frequencies. When applying brain stimulation, it is required to maintain an electric potential oriented in the same direction for a certain period of time. According to the method in Non-Patent Document 2, an electric field oriented in a fixed direction could not be maintained because of high-velocity fluctuations in the direction of the electric field that was generated. For this reason, the method described in Non-Patent Document 2 has the problem that it is difficult to generate an arbitrary electric potential, electric field, or electric current.

Additionally, Non-Patent Document 3 (Boyang Shen, et al., “Design and simulation of superconducting Lorentz Force Electrical Impedance Tomography (LFEIT)”. Physica C: Superconductivity and Its Application, 524, 5-12, 2016) describes a method for generating an electric current by means of electromagnetic induction, by vibrating an article with ultrasonic waves in the presence of a static electric field. The method described in Non-Patent Document 3 had the problem that the direction of the generated electric current varies at high velocity, therefore making it difficult to generate an electric potential/electric current in a fixed direction. Accordingly, conventional methods had the problem that an electric potential or an electric current could not be localized at a desired position, or an electric potential, an electric field or an electric current could not be maintained for a desired period of time.

### SUMMARY OF THE INVENTION

An example of a purpose of the present disclosure is to provide an electric potential generation method, an electric potential generation apparatus, and a program that can use ultrasonic waves and a magnetic field to generate an electric potential having a desired spatial pattern inside an article, and to maintain the electric potential for a desired period of time.

An example of an embodiment disclosed herein is an electric potential generation method that includes generating a magnetic field having an intensity based on an arbitrary first temporal pattern in a region including a target position inside an object, using a magnetic field formation unit that generates a magnetic field; generating one or more ultrasonic waves in which respective parameters of direction, frequency, amplitude, and phase are separately adjusted, by separately controlling one or more wave sources: generating a force having a desired direction, intensity, and shape in accordance with the first temporal pattern so as to move a prescribed region of the object at the target position based on the force, by irradiating the target position with the one or more ultrasonic waves; and generating an arbitrary electric potential based on the magnetic field in the prescribed region.

### BRIEF DESCRIPTION OF THE DRAWINGS

FIG. 1 is a block diagram illustrating the structure of a force field generation apparatus according to an embodiment.

FIG. 2 is a diagram illustrating the structure of an ultrasonic wave output unit.

FIG. 3 is a diagram illustrating the principles by which ultrasonic plane waves are generated.

FIG. 4 is a diagram schematically illustrating the principles by which ultrasonic waves are combined at a target position to generate a force.

FIG. 5 is a perspective view illustrating the principles by which an electric potential is generated inside a conductor.

FIG. 6 is a plan view illustrating the principles by which an electric potential is generated inside a conductor.

FIG. 7A to FIG. 7C are diagrams illustrating an electric potential generated inside a conductor by ultrasonic vibrations.

FIG. 8A is a diagram indicating the time average of the pressure generated inside a conductor by ultrasonic vibrations.

FIG. 8B is a diagram indicating the time average of the velocity generated inside a conductor by ultrasonic vibrations.

FIG. 8C is a diagram indicating the time average of the electric potential generated inside a conductor by ultrasonic vibrations.

FIG. 9A to FIG. 9C are diagrams illustrating an electric potential generated in a conductor when displacement exceeding the ultrasonic vibrations is applied.

FIG. 10A is a diagram indicating the time average of the pressure generated in a conductor when displacement exceeding the ultrasonic vibrations is applied.

FIG. 10B is a diagram indicating the time average of the velocity generated in a conductor when displacement exceeding the ultrasonic vibrations is applied.

FIG. 10C is a diagram indicating the time average of the electric potential generated in a conductor when displacement exceeding the ultrasonic vibrations is applied.

FIG. 11 is a diagram illustrating the principles for generating torsional vibrations at a target position.

FIG. 12A is a diagram indicating the velocity generated at the target position based on the torsional vibrations.

FIG. 12B is a diagram indicating an electric potential distribution generated in the vicinity of the target position.

FIG. 12C is a perspective view indicating an example of an acoustic lens.

FIG. 13 is a diagram indicating the pressure of ultrasonic waves for generating torsional vibrations.

FIG. 14 is a diagram indicating an electric potential distribution generated in the periphery of the target position based on the torsional vibrations.

FIG. 15 is a diagram indicating a resonance frequency of an object.

FIG. 16 is a diagram indicating the pressure of ultrasonic waves when the resonance frequency is applied.

FIG. 17 is a diagram indicating an amplitude when the resonance frequency is applied at the target position.

FIG. 18 is a diagram indicating an electric potential that is generated when the resonance frequency is applied at the target position.

FIG. 19 is a diagram indicating an electric potential distribution generated in the periphery of the target position when the resonance frequency is applied to the target position.

FIG. 20 is a flow chart indicating the flow of processes in the electric potential generation method.

### DETAILED DESCRIPTION OF THE INVENTION

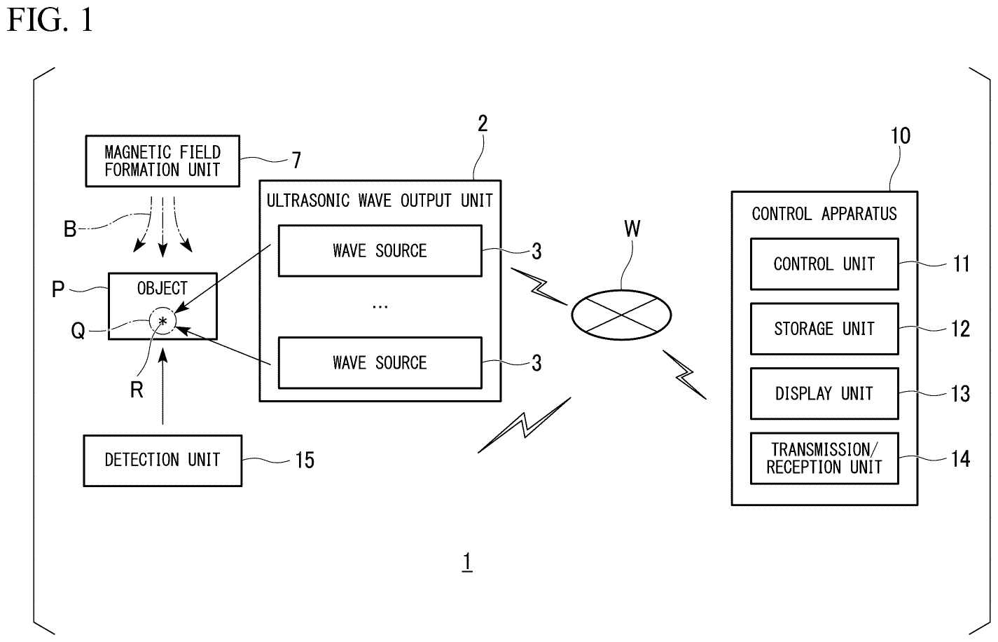

As illustrated in FIG. 1, the electric potential generation apparatus 1 is provided with a magnetic field formation unit 7 for generating a magnetic field, an ultrasonic wave output unit 2 provided with one or more wave sources for generating ultrasonic waves, a control apparatus 10 for controlling the ultrasonic wave output unit 2 and the magnetic field formation unit 7 in a linked manner, and a detection unit 15 for detecting a target position R in an object P. The ultrasonic wave output unit 2 has, for example, an acoustic lens and one or more wave sources 3 for generating ultrasonic waves. Each wave source 3 is disposed at a different position. The structure of the wave sources 3 will be explained below.

The magnetic field formation unit 7 is provided, for example, with one or more electromagnetic coils for generating magnetic fields. The magnetic field formation unit 7 generates intense magnetic fields based on an arbitrary first temporal pattern and an arbitrary first spatial pattern by arbitrarily adjusting the direction, frequency, and intensity of the magnetic field generated by each of the electromagnetic coils in a region including the target position R. The magnetic field formation unit 7 may generate a static magnetic field, or may generate an AC magnetic field. The magnetic field formation unit 7 may, for example, be an MRI (Magnetic Resonance Imaging) apparatus in the case in which the object P is a human body.

The detection unit 15 detects a target position inside the object P. The detection unit 15 may, for example, be an MRI apparatus in the case in which the object P is a human body. In this case, the detection unit 15 may be the magnetic field formation unit 7. The detection unit 15 may be an ultrasonic probe used for diagnosis. The detection unit may be of any type as long as it can measure the target position R inside the object P. Additionally, the detection unit 15 may detect the intensity of the magnetic field at the target position R. The detection unit 15 does not always require to be provided.

The control apparatus 10 is, for example, provided with a control unit 11 for controlling the ultrasonic wave output unit 2 and the magnetic field formation unit 7 in a linked manner based on detected values from the detection unit 15, a storage unit 12 in which data required for control is stored, a display unit 13 for displaying information for executing control, and a transmission/reception unit 14 for transmitting and receiving signals with respect to the detection unit 15, the magnetic field formation unit 7, and the ultrasonic wave output unit 2.

The control apparatus 10 may be connected to the detection unit 15, the magnetic field formation unit 7, and the ultrasonic wave output unit 2 across a network W. The network W is, for example, composed of a public network, a LAN, a WAN, etc. The network W may be composed of various types of connections, such as those using cables or wireless. The network W may also perform near-field communication.

The control apparatus 10, for example, acquires detected values from the detection unit 15. The control apparatus 10 is constituted, for example, by an information processing apparatus such as a personal computer, a tablet terminal, a smartphone, etc. The control apparatus 10 may be a server apparatus connected to the network W. The control apparatus 10 may be configured to operate the ultrasonic wave output unit 2, the magnetic field formation unit 7, and the detection unit 15 in a linked manner over the network W, and may be configured so as to be integrated with the ultrasonic wave output unit 2, the magnetic field formation unit 7, and the detection unit 15.

The control apparatus 10, for example, acquires measurement data from the detection unit 15 via the transmission/reception unit 14. The transmission/reception unit 14 is, for example, a communication interface capable of transmitting and receiving data. The transmission/reception unit 14 stores acquired data in the storage unit 12. The transmission/reception unit 14, as mentioned below, outputs controls signals from the control unit 11 to the magnetic field formation unit 7 and the ultrasonic wave output unit 2. The storage unit 12 is, for example, a non-transitory storage apparatus constituted by a hard disk drive (HDD), a flash memory, etc. The storage unit 12 may be provided integrally with or separately from the control apparatus 10, and may be a server apparatus connected to the network W.

The measurement data stored in the storage unit 12 is read out by the control unit 11. The control unit 11, for example, calculates the relative positional relationship between the respective wave sources 3 and the target position R based on the measurement data. The control unit 11, for example, based on the measurement data, calculate, the coordinates of the respective wave sources 3 with reference to a preset origin position, and also calculates the coordinates of the target position R. The control unit 11 separately controls the magnetic field formation unit 7 and the one or more wave sources 3 based on the coordinates of the target position R.

The control unit 11 generates a magnetic field B in a region including the target position R inside the object P. The control unit 11, for example, calculates the change over time in the magnetic field B based on parameters such as the intensity, direction, frequency, and phase of the magnetic field B at a target position R based on the measurement data at the target position R, the position of the magnetic field formation unit 7, and control information for controlling the magnetic field formation unit 7. The control unit 11 calculates the state of the magnetic field B based on a first temporal pattern at the target position R. The first temporal pattern, for example, relates to an amount of change in the magnetic field B including parameters that change over time. The first pattern may, for example, be a pattern for generating a static magnetic field, or may be a pattern for generating an AC magnetic field. The control unit 11 controls the magnetic field formation unit 7 to generate a magnetic field B having an intensity based on an arbitrary first temporal pattern in a region including the target position R inside the object P.

The control unit 11, for example, separately adjusts the respective parameters of direction, frequency, amplitude, and phase of the respective ultrasonic waves output from the one or more wave sources 3, and causes each of the one or more wave sources 3 to generate ultrasonic waves. The control unit 11 separately controls the one or more wave sources 3 to generate one or more ultrasonic waves for which the respective parameters of direction, frequency, amplitude, and phase are separately adjusted. By irradiating the target position R with ultrasonic waves, the control unit 11 generates, in a prescribed region including the target position R inside the object R a force having a desired direction, intensity, and shape in accordance with the state of the magnetic field based on the first temporal pattern at the target position R. The prescribed region including the target position R inside the object P is, for example, in the case in which the object P is a human body, a lump in human tissue within a prescribed distance range from the target position R. The prescribed region Q is an electrical conductor composed of cell tissue, materials, etc. constituting the object P.

As a result thereof, the prescribed region Q at the target position R in the object P moves from the target position R based on the generated forces. At this time, a magnetic field B based on the first temporal pattern is generated in the prescribed region Q. Therefore, a Lorentz force due to electromagnetic induction is generated in the prescribed region Q based on the relationship between the direction of the velocity generated in the prescribed region Q and the direction and change over time of the magnetic field. An electric potential, an electric field, or an electric current is generated in the prescribed region Q in accordance with the local electrical conductivity or dielectric constant based on this Lorentz force.

The control unit 11 adjusts the movement amount, the movement direction, the movement time, etc. of the prescribed region Q at the target position R by adjusting the magnetic field B and the ultrasonic waves in order to generate a desired electric potential and electric current in the prescribed region Q. The control unit 11 controls the ultrasonic wave output unit 2 and the magnetic field formation unit 7 to irradiate the target position R with one or more ultrasonic waves. The control unit 11 generates forces with desired directions, intensities, and shapes in the prescribed region Q in accordance with the first temporal pattern, moves the prescribed region Q at the target position R, and generates an arbitrary electric potential based on the magnetic field B in the prescribed region Q. The control unit 11, for example, generates an image indicating the generated electric potential and the state of forces based on the generated ultrasonic waves, and cause a display unit 13 to display the image.

The display unit 13 is, for example, a display apparatus constituted by a liquid crystal display, an organic EL display, etc. The display unit 13 may, for example, be constituted by a touch panel, and may be an operation unit for inputting operation information for operating the control apparatus 10. The operation unit may also be separately provided.

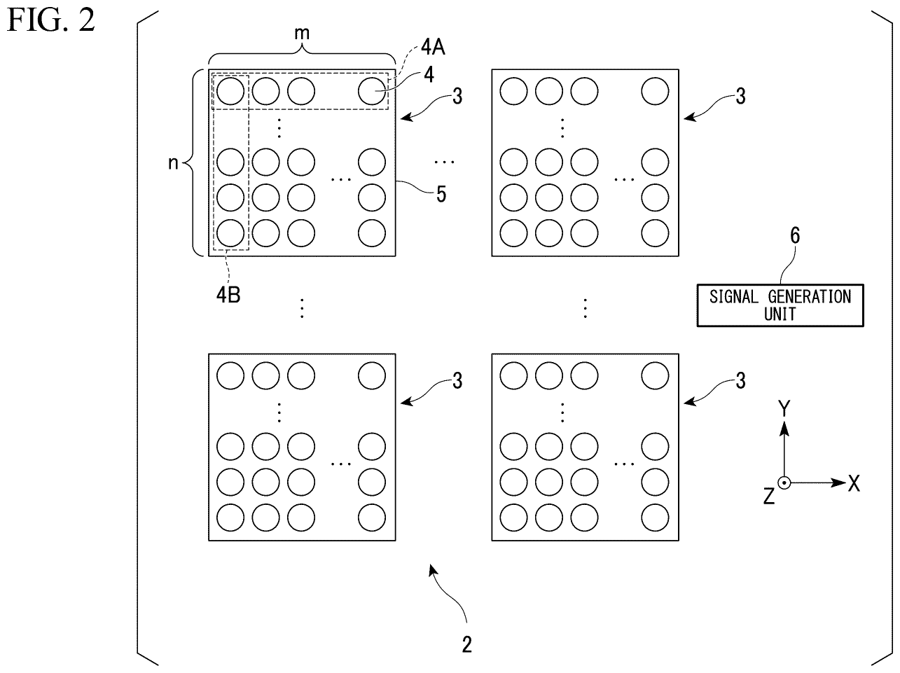

As illustrated in FIG. 2, the ultrasonic wave output unit 2 is constituted by a plurality of wave sources 3 disposed at different positions, and a signal generation unit 6 for controlling the wave sources 3. Each wave source 3 is formed so as to be capable of inputting ultrasonic waves to the object P. For example, in the case in which the object P is a human body, each wave source 3 may be formed in the shape of a pad that can be affixed to the object P. The wave sources 3 may be arrayed on a single substrate 5. The wave sources 3 are electrically connected to the signal generation unit 6.

Each wave source 3 is a phased-array-type ultrasonic transducer constituted by multiple ultrasonic vibrators 4. Each wave source 3 is provided with, for example, multiple ultrasonic vibrators 4 arranged in matrix form. The multiple ultrasonic vibrators 4 are, for example, arranged in m×n (where m and n are arbitrary natural numbers) matrix form on a substrate 5. On the substrate 5, a single column of ultrasonic vibrator units 4A consisting of in ultrasonic vibrators 4 is formed in a first direction (the X-axis direction in the drawing). A single column of ultrasonic vibrator units 4B consisting of n ultrasonic vibrators 4 is formed in a second direction (the Y-axis direction in the drawing) orthogonal to the array direction of the ultrasonic vibrator units 4A.

The array method for the multiple ultrasonic vibrators 4 is an example, and other array methods may be used. Additionally, the numbers mu and n may be different in each wave source 3. In each wave source 3, the number of the multiple ultrasonic vibrators 4 to be controlled can be adjusted, and the numbers n and n can be adjusted. In the case in which multiple ultrasonic vibrators 4 arranged in a matrix form are arrayed on a single substrate 5, the multiple wave sources 3 may be constituted by dividing the substrate 5 into multiple regions of arbitrary sizes, and controlling each of the regions separately. The respective wave sources 3 may be disposed adjacent to each other, or may be disposed at different locations.

Each ultrasonic vibrator 4 is electrically connected to a signal generation circuit (not illustrated) separately provided in the signal generation unit 6. Each ultrasonic vibrator 4 has a vibrator for vibrating based on RI power output from the signal generation circuit, and outputs ultrasonic vibrations based on vibrations of the vibrator. Each signal generation circuit is separately controlled by the control unit 11. The control unit 11 separately controls each signal generation circuit to separately adjust the respective parameters of frequency, amplitude, and phase of the ultrasonic waves output from the respective ultrasonic vibrators 4.

The control unit 11, for example, generates an ultrasonic beam that propagates in the form of a beam, in which the frequencies and amplitudes of the multiple ultrasonic vibrators 4 constituting the respective wave sources 3 are made the same, and separately adjusts the phases of the respective ultrasonic vibrators 4 to adjust the propagation direction of the ultrasonic beam. The ultrasonic beam propagates by forming plane waves of compressional waves. The control unit 11 may adjust the phase and polarization direction of the ultrasonic waves output from the respective wave sources 3 to generate ultrasonic waves based on circularly polarized waves or spiral beams.

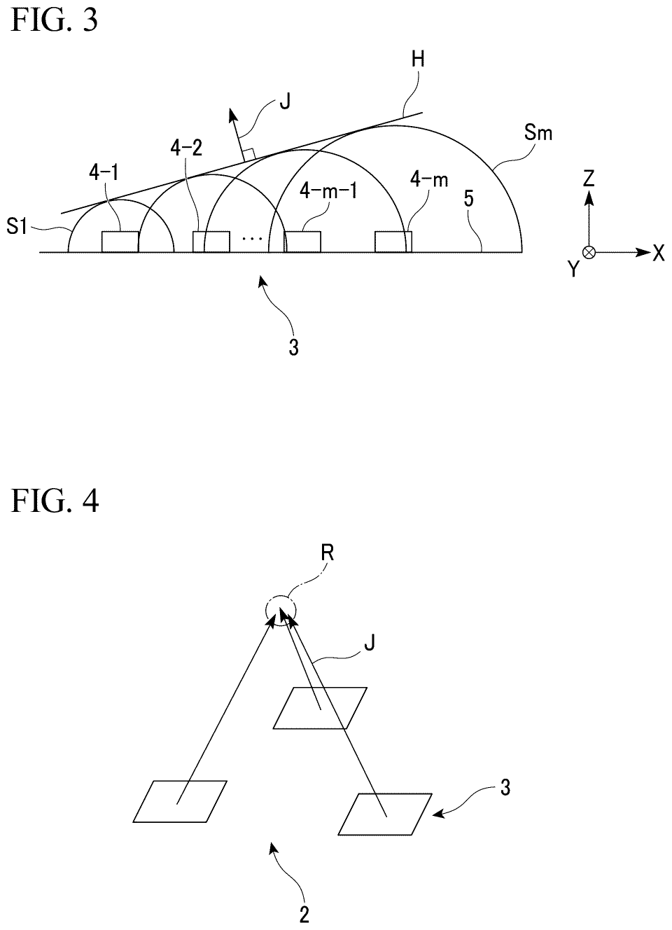

As illustrated in FIG. 3, ultrasonic waves that are spherical waves Sm are output from each ultrasonic vibrator 4-m in the ultrasonic vibrator unit 4A. At this time, when the phases of the ultrasonic waves output from adjacent ultrasonic vibrators 4-m are delayed by prescribed amounts in a first direction, a plane wave J is formed at the envelope H at which the ultrasonic spherical waves Sm output from the respective ultrasonic vibrators 4-m form an equiphase surface. This plane wave J propagates in a direction orthogonal to the envelope line H.

In a similar way, delaying the phases of the ultrasonic waves output from the adjacent ultrasonic vibrators 4-n (not illustrated) in the ultrasonic vibrator unit 4B (see FIG. 2), which is in the direction orthogonal to the ultrasonic vibrator unit 4A, by prescribed amounts, the propagation direction of the formed plane waves J can be adjusted. Based on the abovementioned control method, the wave source 3 can form an ultrasonic beam of plane waves that propagate in an arbitrary direction in three dimensions. Additionally, the wave source 3 may adjust the phase and polarization directions of ultrasonic waves output from the respective ultrasonic vibrators 4 to generate ultrasonic waves that are circularly polarized waves or spiral beams.

Aside from being configured as a phased array in which multiple ultrasonic vibrators 4 are arrayed, the wave sources 3 may be configured so as to use an acoustic prism to make the ultrasonic waves propagate in a specific direction. The ultrasonic vibrator unit 4A may use an acoustic lens to generate ultrasonic waves that are circularly polarized waves or spiral beams. Multiple wave sources 3 may be an acoustic lens that focuses ultrasonic waves output from a single sound source so as to generate a focal point at the target position. That is, the multiple wave sources 3 may be respective microelements that have been computationally modeled on an acoustic lens that outputs ultrasonic waves from a single wave source 3 via the acoustic lens. In this case, the acoustic lens may be configured to output ultrasonic waves with controlled acoustic pressure and phases based on the ultrasonic waves output from the respective microelements serving as the multiple wave sources 3.

That is, the acoustic lens may have a shape that is designed so as to computationally adjust the parameters of the respective ultrasonic waves output from the respective microelements serving as the multiple wave sources 3, thereby focusing the ultrasonic waves on the target position, generating a continuous sound source by taking the limit of each microelement, and designing the shape so as to generate a desired force at the target position. An acoustic lens that is designed in this way may be configured to separately adjust the respective parameters of direction, frequency, amplitude, and phase of the respective ultrasonic waves in the respective microelements serving as the multiple wave sources when outputting ultrasonic waves, to generate multiple ultrasonic waves having different frequencies from the respective microelements, to combine multiple ultrasonic waves at the target position in the object to generate a desired force. The processes in the above-mentioned control apparatus 10 may include computations for controlling the ultrasonic wave output unit 2 to generate a combined wave at the target position through an acoustic lens.

As illustrated in FIG. 4, the control unit 11 can separately adjust the respective parameters of frequency, amplitude, and phase of the ultrasonic waves output front multiple ultrasonic vibrators 4 provided in multiple wave sources 3 to output multiple plane waves J towards a target position R. As a result thereof, the control unit 11 can generate multiple ultrasonic waves having different frequencies from the multiple wave sources 3, combine multiple ultrasonic waves at the target position R inside an article, and generate a force having a desired direction, intensity, and shape based on a desired temporal pattern.

Hereinafter, the principles for generating an arbitrary electric potential in a prescribed region Q of a target position R will be explained.

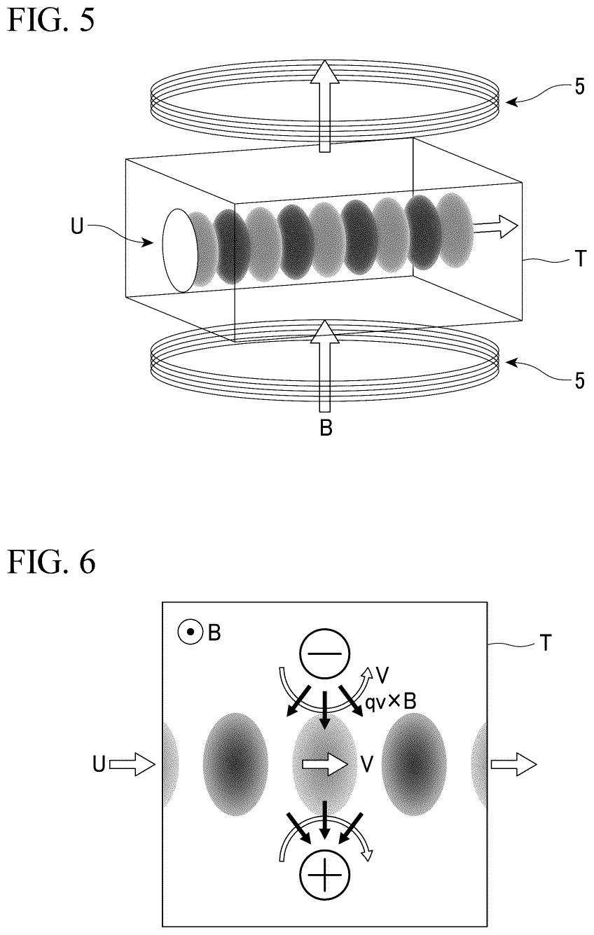

As illustrated in FIG. 5 and FIG. 6, when there are ultrasonic waves U in a medium T, the medium T vibrates in the propagation direction of the ultrasonic waves U at the frequency of the ultrasonic waves U. The velocity vector of the medium T is represented by v (see FIG. 6). The case in which a spatially uniform static magnetic field is applied to the medium T will be considered. In this case, in the medium T vibrating based on the ultrasonic waves U, a Lorentz force of qv×B will be exerted per charge q (see FIG. 6), where B represents the vector of the magnetic field and x represents a vector product. The necessary and sufficient conditions for the Lorentz force acting on the medium T to be non-zero is for v×B to be non-zero, i.e., for v to not be parallel to B, and thus to have a component orthogonal to the magnetic field direction of B.

In the medium T, the Lorentz force generated based on the ultrasonic waves U causes the charge distribution to change. In the example in FIG. 6, at a certain moment in the medium T, a low-potential portion is generated in the upper portion and a high-potential portion is generated in the lower portion as viewed in the direction of the page. When the magnetic field B is spatially uniform, an electric potential is generated based on a component of the vorticity vector of v in a direction orthogonal to the magnetic field.

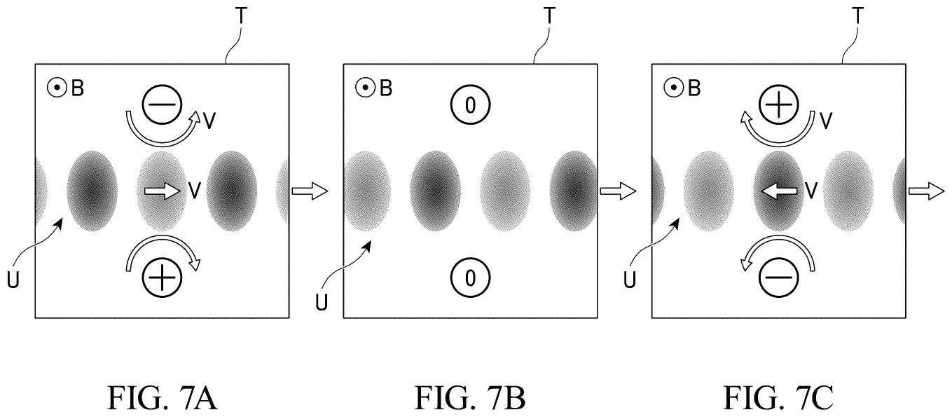

As illustrated in FIG. 7A to FIG. 7C, the phases of the ultrasonic waves U change with the passage of time, and the velocity v of the medium T vibrating based on the ultrasonic waves U also changes in accordance therewith. Thus, the electric potential V generated in the medium T changes in accordance with the velocity v.

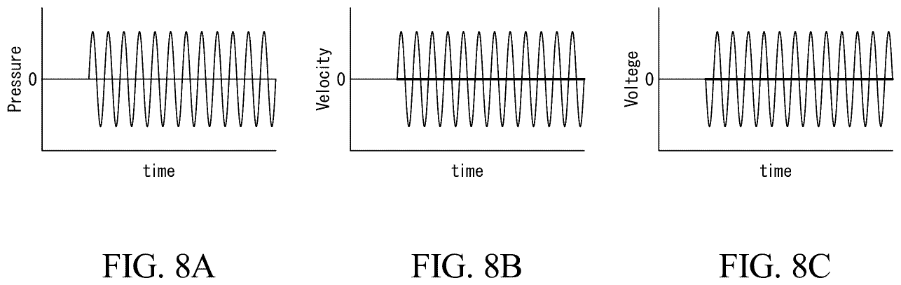

As illustrated in FIG. 8A to FIG. 8C, in the case in which the medium T only vibrates based on the ultrasonic waves U and there is no net movement, the time average of the velocity v is zero (see FIG. 8B). At this time, the time average of the electric potential V generated in the medium T is also zero (see FIG. 8C). When there are non-linear effects or acoustic waves are absorbed in the medium T, there are cases in which the medium T is accelerated in a certain direction (see, for example, Non-Patent Document 4 (Kathy Nightingale, et al., “Acoustic Radiation Force Impulse (ARFI) Imaging: A Review”. Current Medical Imaging. Volume 7. Issue 4, pages 328-339, 2011)).

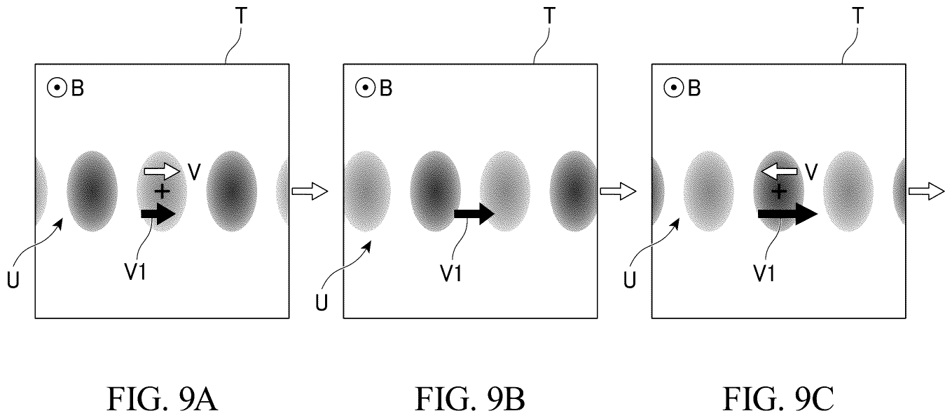

When the medium T is accelerated in a certain direction while in a static magnetic field, as illustrated in FIG. 9A to FIG. 9C, the time average V1 of the velocity v becomes non-aero.

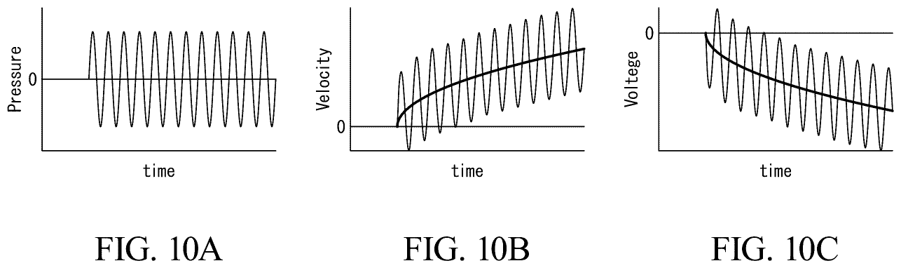

As illustrated in FIG. 10A to FIG. RC, along with the time average V1 of the velocity v, the time average of the electric potential also becomes non-zero. According to these principles, when a desired position inside a conductor, in which a magnetic field is generated, is irradiated with ultrasonic waves so as to cause movement such that the time average of the velocity becomes non-zero, an electric potential that is permanent in time is generated in the conductor. That is, an electric potential having a time average that is non-zero can be generated in the conductor by adjusting the radiation pattern of ultrasonic waves so that movement that is not parallel to the direction of the magnetic field occurs at the desired position in the conductor.

Furthermore, an electric potential that lasts for a desired time period, having a desired spatial pattern and temporal pattern, can be generated in an article by adjusting the radiation patterns of the ultrasonic waves. Additionally, the radiation pattern of the ultrasonic waves can be adjusted in accordance with the temporal pattern and the spatial pattern of the magnetic field, or the temporal pattern and the spatial pattern of the magnetic field can be adjusted in accordance with the movement of the conductor occurring based on the radiation pattern of the ultrasonic waves.

That is, the control unit 11 in the electric potential generation apparatus 1 controls the magnetic field formation unit 7 to generate a magnetic field B based on an arbitrary temporal pattern (first temporal pattern) at the target position R. The temporal pattern of the magnetic field B may be a DC pattern, or may change over time, such as an AC pattern. The control unit 11 controls the ultrasonic wave output unit 2 to irradiate the target position R with ultrasonic waves so as to generate a force, in which a direction is adjusted in accordance with the generated direction of the magnetic field B, thereby moving the prescribed region Q in the direction of the force. As a result thereof, an electric potential having an arbitrary temporal pattern (second temporal pattern) can be generated at an arbitrary time in the prescribed region Q.

The temporal pattern of the electric potential may be a DC pattern, or may change over time, such as an AC pattern, a pulse, a shock voltage, etc. Due to the above-mentioned configuration, the electric potential generation apparatus 1 can, for example, generate an arbitrary electric potential, such as an electric potential required for firing a neuron, a membrane potential, an oxidation reduction potential, etc., at the target position R in a living body. Additionally, the electric potential generation apparatus 1 may generate an AC potential or a pulse potential at the target position R in the living body, or may generate Joule heat based on the electric potential (electric current).

When generating a DC electric potential in a conductor in which a static magnetic field is generated at a desired time, it is required to continuously move a prescribed region Q including the target position R in the object P. The DC electric potential mentioned here includes not only an electric potential that is constant over time, but also an electric potential with a non-zero time average (the same hereinafter). When a force is applied in a prescribed direction at the target position R in the object P based on ultrasonic waves, the prescribed region Q is accelerated in the direction in which the force is applied, moves a prescribed distance based on the reaction force generated by elastic deformation, then stops. At this time, an electric potential is generated in the prescribed region Q during the time required for movement.

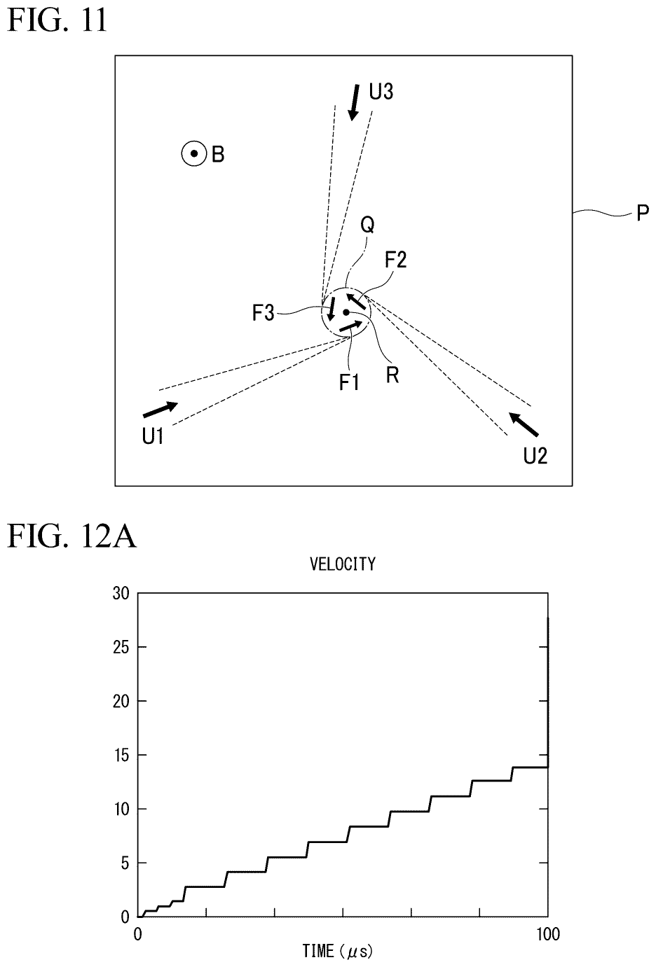

As illustrated in FIG. 11, the control unit 11 controls the magnetic field formation unit 7 to generate a static magnetic field. In the example in FIG. 11, the magnetic field B is generated from the back side of the page surface towards the front side. When a constant magnetic field B is automatically generated in the magnetic field formation unit 7, the control unit 11 does not always require to control the magnetic field formation unit 7 over time. The control unit 11 controls the ultrasonic wave output unit 2 to irradiate a target position R (strictly speaking, a position slightly offset from the target position R: the same hereinafter) with the ultrasonic waves U.

The control unit 11 controls the ultrasonic wave output unit 2 to radiate first ultrasonic waves U1 from a first wave source 3. The control unit 11 generates a first force F1 so as to generate a velocity component v in the same direction in the prescribed region Q over a period of time that is long in comparison to the vibration period of the first ultrasonic waves U1 at the target position R. At this time, the control unit 11 generates the first force F1 in a direction substantially orthogonal to the direction of the magnetic field B to move the prescribed region Q in accordance with the direction of the first force. The control unit 11 generates a DC electric potential in the prescribed region Q for a prescribed period of time required for movement (see FIG. 9A to FIG. 9C and FIG. 10A to FIG. 10C).

The control unit 11 controls the ultrasonic wave output unit 2 in a manner similar to the above-mentioned control to radiate second ultrasonic waves U2 from a second wave source 3 at timings until the prescribed region Q moving based on the first force F1 stops. The control unit 11 generates a second force F2 so as to generate a velocity component v in the same direction in the prescribed region Q over a period of time that is long in comparison to the vibration period of the second ultrasonic waves U2 at the target position R. At this time, the control unit 11 generates the second force F2 in a direction substantially orthogonal to the direction of the magnetic field B to move the prescribed region Q in accordance with the direction of the second force. The control unit 11 generates a DC electric potential in the prescribed region Q based on the second force F2, as with the first force F1, for a prescribed period of time required for movement.

The control unit 11 controls the ultrasonic wave output unit 2 in a manner similar to the above-mentioned control to radiate third ultrasonic waves U3 from a third wave source 3 at timings until the prescribed region Q moving based on the second force F2 stops. The control unit 11 generates a third force F3 so as to generate a velocity component v in the same direction in the prescribed region Q over a period of time that is long in comparison with the vibration period of the third ultrasonic waves U3 at the target position R. At this time, the control unit 11 generates the third force F3 in a direction substantially orthogonal to the direction of the magnetic field B to move the prescribed region Q in accordance with the direction of the third force. The control unit 11 generates a DC electric potential in the prescribed region Q based on the third force F3, as with the first force F1, for a prescribed period of time required for movement.

The control unit 11 executes control for radiating multiple ultrasonic waves as described above to continuously move the prescribed region Q and to generate a IX electric potential in the prescribed region Q for an arbitrary time period. The control unit 11 may radiate the multiple ultrasonic waves with time delays, or may radiate them at the same time. The control unit 11 may repeatedly execute control or may implement control a single time to radiate the multiple ultrasonic waves. In the examples in FIG. 11, the case in which there are three wave sources 3 is indicated. However, there may be three or more wave sources 3. That is, the control unit 11 outputs multiple ultrasonic waves U from different directions towards the target position R based on multiple wave sources 3 disposed at different positions. The control unit 11 combines the multiple ultrasonic waves U at the target position R to generate a rotating force in a planar direction substantially orthogonal to the direction of the magnetic field B, thereby generating torsional vibrations in the prescribed region Q. As a result thereof, the control unit 11 generates a DC electric potential in the prescribed region Q for a prescribed time period that is desired.

In the example in FIG. 11, a force rotating counterclockwise as viewed in the direction opposite to the magnetic field direction is generated. At this time, the rotation direction may be clockwise. The control unit 11 generates a magnetic field that is a static magnetic field at the target position, generates a force F rotating in a planar direction substantially orthogonal to the direction of the magnetic field B at the target position R, generates torsional vibrations having rotation components (∇×v) with respect to the magnetic field B in the prescribed region Q in the direction of the force, and generates a DC electric potential in the prescribed region Q for a prescribed time period.

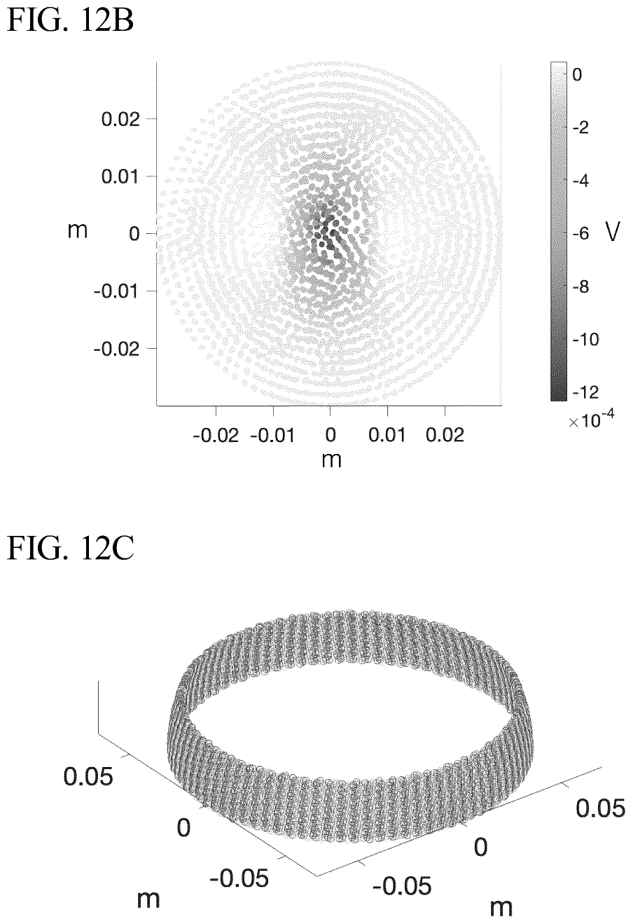

FIG. 12A indicates estimated values of the velocity generated in the prescribed region Q based on simulations. FIG. 12B indicates estimated values of the electric potential distribution generated in the vicinity of the target position R based on simulations, where the simulations were performed by assuming that the medium of the object P is a brain, and by setting the values indicated below as the parameters. Ultrasonic wave period: 0.15 μs; ultrasonic wave frequency: 6.7 MHz; number of wave sources 3: 24; directions of wave sources 3: 24 directions; ultrasonic wave radiation time: 100 μs; velocity measurement position from target position R: 4 mm; ultrasonic wave peak energy: 1440 W/cm²; density of medium: 10³ kg/m³; velocity of sound: 1500 m/s; absorption factor of medium: 0.3 dB/MHz/cm; Young modulus of medium: 4 kPa; Poisson ratio of medium: 0.4; magnetic field intensity at target position: 3.5 T; electric potential measurement timing: 100 μs from starting radiation of ultrasonic waves.

As illustrated, in the prescribed region Q including the target position R, the velocity is generated and a DC electric potential is generated. In this way, multiple ultrasonic waves output from multiple wave sources 3 disposed at different positions are combined with respect to the target position R in the object P in which a static magnetic field has been generated, to generate a rotating force in a planar direction substantially orthogonal to the direction of the magnetic field, thereby it is possible to generate a DC electric potential in the prescribed region Q. The prescribed region Q may cause to rotate not only based on ultrasonic waves radiated from multiple directions, but also based on sound waves output from the ultrasonic wave output unit 2.

For example, sound waves output from the ultrasonic wave output unit 2 can be input to an acoustic lens to generate vortex sound waves, a force rotating in a planar direction substantially orthogonal to the direction of the magnetic field can be generated based on the vortex sound waves at the target position R, torsional vibrations can be generated in the prescribed region Q, and a DC electric potential can be generated in the prescribed region Q. The acoustic lens can be formed in the shape of a three-dimensional spiral or may be formed in the shape of a Fresnel lens. Additionally, the acoustic lens can be formed based on acoustic metamaterials. An example of an acoustic lens is illustrated in FIG. 12C. In the example in FIG. 12C, the thickness of the lens is indicated by differences in color. The vortex sound waves may be generated by controlling multiple wave sources 3 arranged in an array.

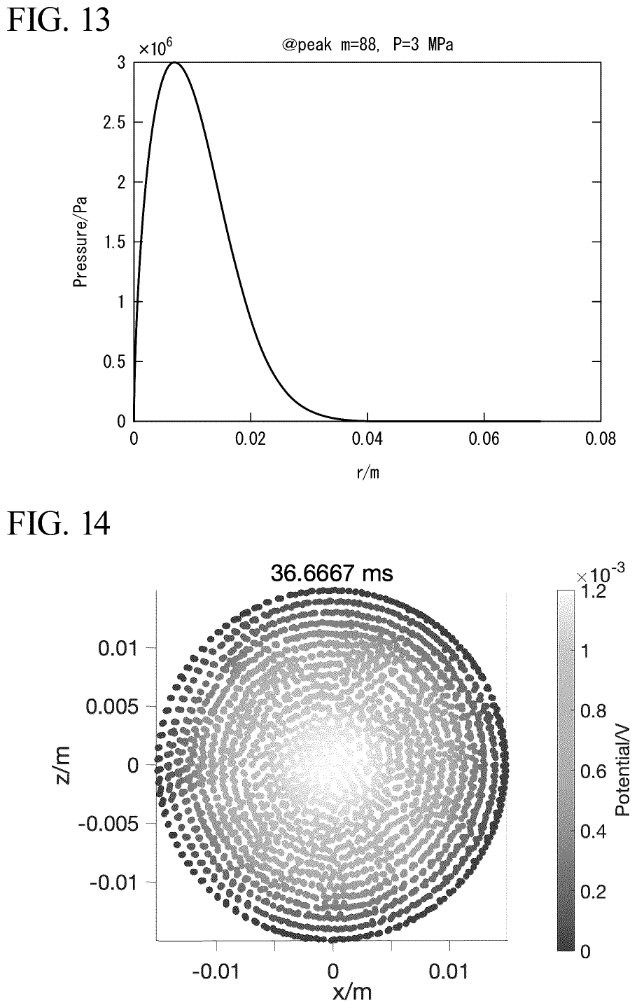

FIG. 13 indicates an example of the acoustic pressure distribution of vortex sound waves input to the prescribed region Q. FIG. 14 indicates the electric potential distribution generated in the prescribed region Q based on the vortex sound waves in FIG. 13, where the respective parameters relating to the ultrasonic waves are set to the values below. Magnetic field intensity at target position R: 3.5 T; peak energy of ultrasonic waves: 600 W/cm², maximum acoustic pressure of ultrasonic waves: 3 MPa; radiation radius of ultrasonic waves: 0.007 m; frequency of ultrasonic waves: 3 MHz. The other parameters are the same as those used in the example in FIG. 11 and FIG. 12. As illustrated in FIG. 14, an electric potential of approximately 1.2 mV is generated in the prescribed region Q. To increase the electric potential generated in the prescribed region Q, the amplitude of the torsional vibrations generated in the prescribed region Q can be increased.

As indicated in FIG. 15, when torsional vibrations are applied to the prescribed region Q with a period matching a period specific to the medium, the prescribed region Q resonates and the amplitude increases. The vibration frequency of the torsional vibrations of an elastic body are determined by the rotation diameter, and the Young modulus, the Poisson ratio and the density of the medium, etc. For this reason, by adjusting the parameters of the ultrasonic waves so as to generate a force that becomes torsional vibrations at a period causing the prescribed region to resonate in accordance with the medium constituting the object P, a DC electric potential that is large relative to the little energy input to the prescribed region Q can be generated for a prescribed time period in the prescribed region Q.

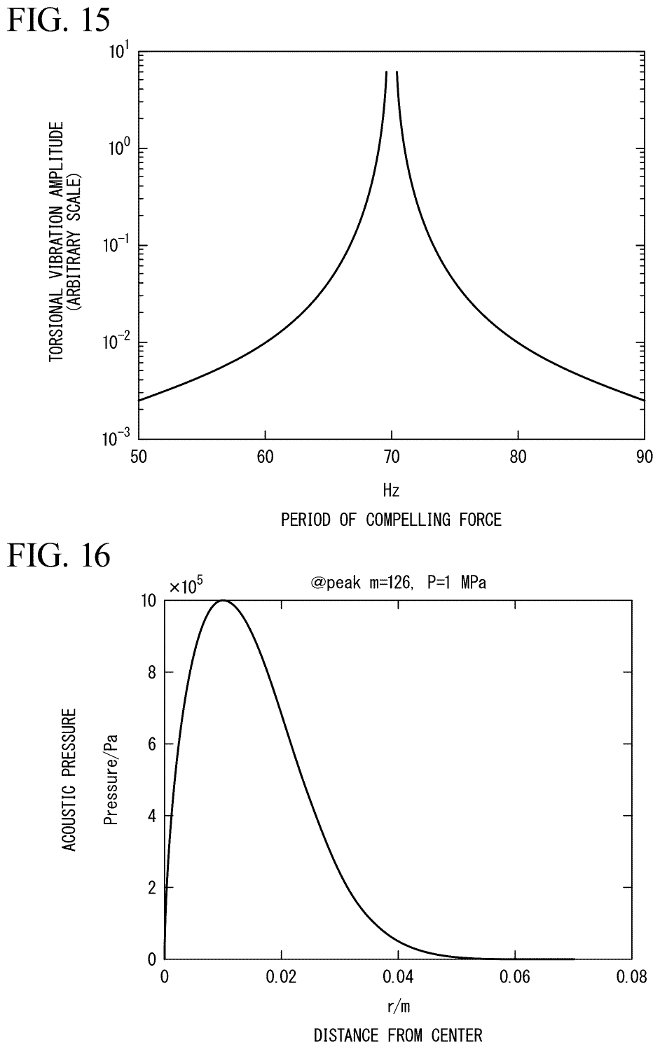

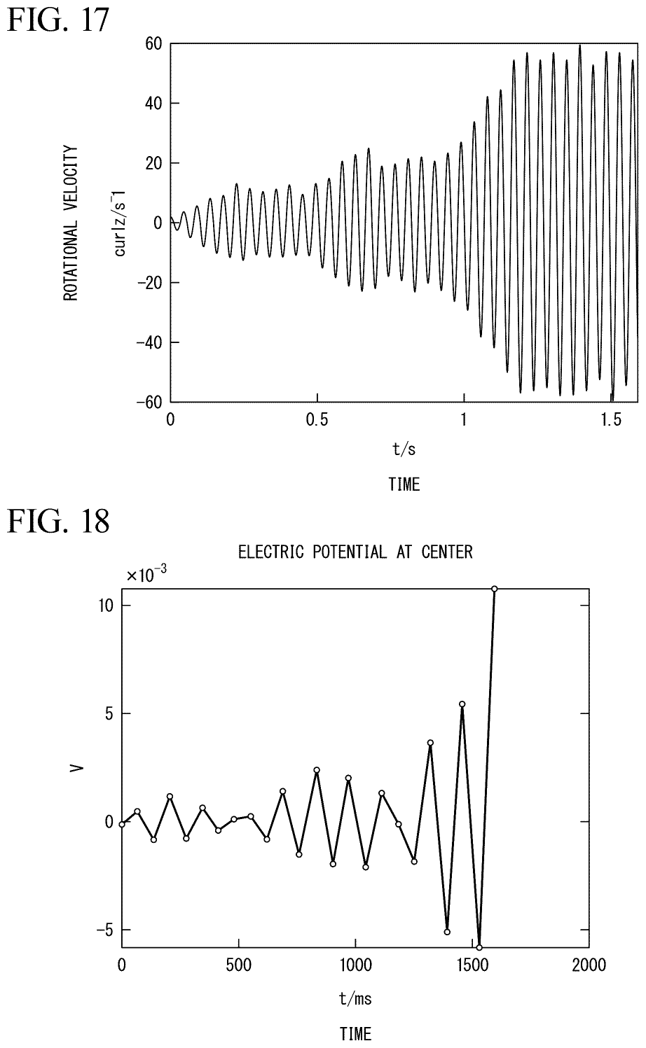

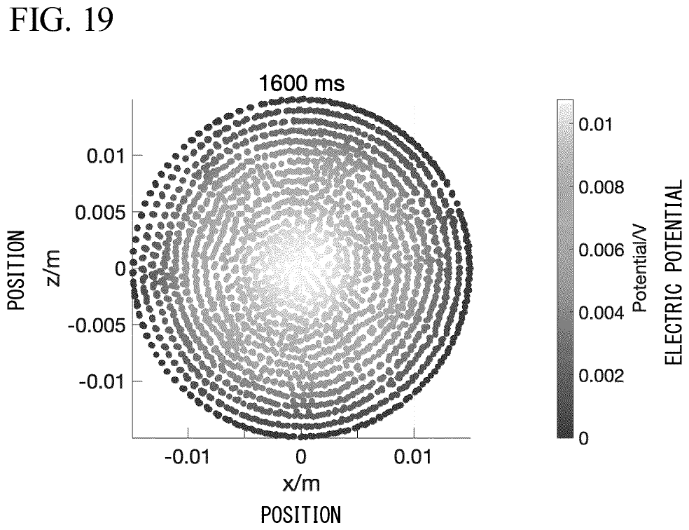

As indicated in FIG. 16 to FIG. 19, when torsional vibrations based on the resonance frequency are generated in the prescribed region Q, a large DC electric potential can be generated for a prescribed time period in the prescribed region Q. In the examples indicated in FIG. 16 to FIG. 19, the respective parameters relating to ultrasonic waves are set to the values below. Magnetic field intensity at target position R: 3.5 T; peak energy of ultrasonic waves: 67 W/cm², maximum acoustic pressure of ultrasonic waves: 1 MPa; radiation radius of ultrasonic waves: 0.007 m; frequency of ultrasonic waves: 3 MHz. The other parameters are the same as those used in the example in FIG. 11 and FIG. 12A.

In the example indicated in FIG. 16 to FIG. 19, an electric potential of approximately 11 mV was generated in the prescribed region Q. In the example indicated in FIG. 16 to FIG. 19, the energy of the input ultrasonic waves was approximately 1/10 that of those in the example indicated in FIG. 11 and FIG. 12, yet the electric potential generated in the prescribed region Q increased by approximately a factor of 10, and the energy efficiency increased by more than 80 times. That is, the control unit 11 controls the ultrasonic wave output unit 2 so as to irradiate the target position R with ultrasonic waves, generates a force that becomes torsional vibrations with a period at which the prescribed region Q resonates, and generates a DC electric potential for an arbitrary prescribed time period in the prescribed region Q.

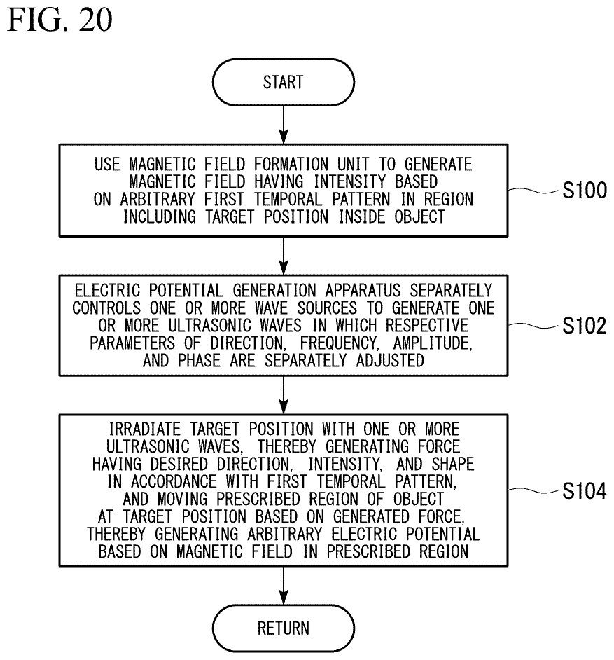

FIG. 20 indicates the flow of processes in the electric potential generation method using the electric potential generation apparatus 1. The electric potential generation apparatus 1 uses the magnetic field formation unit 7 that generates a magnetic field to generate a magnetic field having an intensity based on an arbitrary first temporal pattern, in a region including the target position R inside the object P (step S100). The electric potential generation apparatus 1 separately controls one or more wave sources 3 to generate one or more ultrasonic waves in which the respective parameters of direction, frequency, amplitude, and phase are separately adjusted (step S102). The electric potential generation apparatus 1 irradiates the target position R with the one or more ultrasonic waves, thereby generating a force having a desired direction, intensity, and shape in accordance with the first temporal pattern, and moving a prescribed region Q of the object P at the target position R based on the generated force, thereby generating an arbitrary electric potential based on the magnetic field in the prescribed region Q (step S104).

As mentioned above, according to the electric potential generation apparatus 1, ultrasonic waves and magnetic fields can be used to generate an arbitrary electric potential at an arbitrary position inside a conductor (object P) such as a living body, and this can be applied to a non-invasive therapeutic apparatus. According to the electric potential generation apparatus 1, electric potentials having an arbitrary temporal pattern can be generated at an arbitrary position inside a conductor. Thus, an electric potential, an electric field or an electric current having a desired effect, such as an electric potential for firing a neuron, a membrane potential, or an oxidation reduction potential can be generated. According to the electric potential generation apparatus 1, a DC electric potential can be generated for an arbitrary time period in the prescribed region Q by using ultrasonic waves to continuously move the prescribed region Q in the object P.

According to the electric potential generation apparatus 1, by irradiating the prescribed region Q with a vortex sound wave based on ultrasonic waves, torsional vibrations can be generated in the prescribed region, and an electric potential with a non-zero time average can be continuously generated in the prescribed region Q. According to the electric potential generation apparatus 1, when generating torsional vibrations in the prescribed region Q, an electric potential that is large relative to the little energy that is input can be generated by causing the prescribed region Q to resonate. Additionally, according to the electric potential generation apparatus 1, an electric potential having an arbitrary temporal pattern can be generated at an arbitrary position inside a conductor, and this can be applied to non-destructive repair apparatuses or inspection apparatuses.

The control unit 11 described above is realized by a processor such as a CPU (Central Processing Unit) or a GPU (Graphics Processing Unit) executing a program (software). Some or all of the functional units thereof may be realized by means of hardware such as an LSI (Large-Scale integrated circuit), an ASIC (Application-Specific Integrated Circuit), or an FPGA (Field-Programmable Gate Array), or may be realized by cooperation between software and hardware. The program may be stored in a storage apparatus, such as an HDD (Hard Disk Drive) or flash memory, provided in the storage unit 12, may be stored in a detachable storage medium such as a DVD or CD-ROM, or may be installed in a storage apparatus by a storage medium being loaded in a drive apparatus. Additionally, a program is not necessarily required, and prescribed operations may be executed by configuring a sequential circuit in the control apparatus 10.

The program is executed by having a computer installed in the control apparatus 10 for controlling, in a linked manner, the magnetic field formation unit 7 for generating a magnetic field having an intensity based on an arbitrary first temporal pattern in a region including a target position inside the object P, and an ultrasonic wave output unit 2 provided with one or more wave sources 3 for generating ultrasonic waves, so as to perform the processes below.

The program controls the magnetic field formation unit 7 to generate a magnetic field in a region including the object P. The program controls the ultrasonic wave output unit 2 to generate, from one or more wave sources 3, one or more ultrasonic waves in which respective parameters of direction, frequency, amplitude, and phase are separately adjusted. As a result thereof, a force having a spatial pattern with a desired direction, intensity, and shape in accordance with the first temporal pattern is generated based on the one or more ultrasonic waves with which the target position R is irradiated, and the prescribed region Q of the object P at the target position R is moved based on the generated force, thereby creating an arbitrary electric potential based on the magnetic field in the prescribed region Q.

According to at least one of the exemplary embodiments, for example, the ultrasonic waves and the magnetic field can be used to generate, and to maintain for a prescribed time period, an electric potential having a desired temporal pattern inside an article.

While an embodiment of the present disclosure has been explained above, the present disclosure is not limited to the embodiment described above, and modifications can be made, as appropriate, without departing from the spirit thereof. For example, the electric potential generation apparatus 1 may generate not just torsional vibrations in the prescribed region Q, but also linear vibrations. In this case, the electric potential generation apparatus 1 may control the magnetic field formation unit 7 in accordance with the vibration frequency of the prescribed region Q to generate an AC magnetic field, or may generate a DC electric potential in the prescribed region Q. Additionally, the electric potential generation apparatus 1 may combine ultrasonic waves with different frequencies to generate beats, and may make the prescribed region Q resonate based on the heats.

The present disclosure includes the embodiments below.

[1] An electric potential generation method that includes generating a magnetic field having an intensity based on an arbitrary first temporal pattern in a region including a target position inside an object, using a magnetic field formation unit that generates a magnetic field, generating one or more ultrasonic waves in which respective parameters of direction, frequency, amplitude, and phase are separately adjusted, by separately controlling one or more wave sources: generating a force having a desired direction, intensity, and shape in accordance with the first temporal pattern so as to move a prescribed region of the object at the target position based on the force, by irradiating the target position with the one or more ultrasonic waves; and generating an arbitrary electric potential based on the magnetic field in the prescribed region.

[2] The electric potential generation method according to [1], which includes generating the force having a direction adjusted in accordance with a direction of the magnetic field generated based on the first temporal pattern at the target position so as to move the prescribed region in the direction of the force; and generating an electric potential having an arbitrary second temporal pattern in the prescribed region.

[3] The electric potential generation method according to [1] or [2], which includes generating the force so as to generate a velocity component in the same direction as the prescribed region for a time period that is long in comparison with a vibration period of the ultrasonic waves at the target position; and generating a DC electric potential in the prescribed region for a prescribed time period.

[4] The electric potential generation method according to any one of [1] to [3], which includes generating the force in a direction substantially orthogonal to the direction of the magnetic field to move the prescribed region in the direction of the force; and generating the DC electric potential in the prescribed region for an arbitrary time period.
[5] The electric potential generation method according to any one of [1] to [4], which includes generating the magnetic field that is a static magnetic field at the target position: generating the force, which rotates in a planar direction substantially orthogonal to the direction of the magnetic field at the target position so as to generate torsional vibrations in the prescribed region in the direction of the force; and generating the DC electric potential in the prescribed region for a prescribed time period.

[6] The electric potential generation method according to any one of [1] to [5], which includes outputting a plurality of the ultrasonic waves from different directions towards the target position based on a plurality of the wave sources disposed at different positions; and generating a force rotating in a planar direction substantially orthogonal to the direction of the magnetic field, by combining the plurality of the ultrasonic waves at the target position.

[7] The electric potential generation method according to any one of [1] to [5], which includes making the ultrasonic waves generate vortex sound waves based on an acoustic lens; and generating the three rotating in a planar direction substantially orthogonal to the direction of the magnetic field based on the vortex sound waves at the target position.

[8] The electric potential generation method according to any one of [1] to [5], which includes generating vortex sound waves, by combining the plurality of ultrasonic waves output from the plurality of wave sources disposed at different positions; and generating the force rotating in a planar direction substantially orthogonal to the direction of the magnetic field based on the vortex sound waves at the target position.

[9] The electric potential generation method according to any one of [1] to [9], which includes generating the force so as to generate the torsional vibrations with a period causing the prescribed region to resonate; and generating the DC electric potential in the prescribed region for the prescribed time period.

[10] An electric potential generation apparatus that includes a magnetic field formation unit that generates a magnetic field having an intensity based on an arbitrary first temporal pattern in a region including a target position inside an object; an ultrasonic wave output unit having one or more wave sources that generate ultrasonic waves; and a control apparatus that controls the ultrasonic wave output unit and the magnetic field formation unit in a linked manner; wherein the control apparatus controls the magnetic field formation unit to generate a magnetic field in the region including the object, and controls the ultrasonic wave output unit to generate one or more of the ultrasonic waves in which respective parameters of direction, frequency, amplitude, and phase are separately adjusted, and irradiate the target position with the ultrasonic waves so as to generate a force having a desired direction, intensity, and shape in accordance with the first temporal pattern, move a prescribed region of the object at the target position based on the force, and generate an arbitrary electric potential based on the magnetic field in the prescribed region.

[11] A non-transitory computer-readable storage medium storing a program to be executed by a computer installed in a control apparatus that controls, in a linked manner, a magnetic field formation unit that generates a magnetic field having an intensity based on an arbitrary first temporal pattern in a region including a target position inside an object, and an ultrasonic wave output unit comprising one or more wave sources that generate ultrasonic waves, wherein the program makes the computer control the magnetic field formation unit to generate a magnetic field in the region including the object; and control the ultrasonic wave output unit to execute a process for generating, from the one or more wave sources, one or more of the ultrasonic waves in which respective parameters of direction, frequency, amplitude, and phase are separately adjusted, so as to generate a force having a desired direction, intensity, and shape in accordance with the first temporal pattern based on the one or more ultrasonic waves with which the target position has been irradiated, move a prescribed region of the object at the target position based on the force, and generate an arbitrary electric potential based on the magnetic field in the prescribed region.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An electric potential generation method that comprises:
generating a magnetic field having an intensity based on an arbitrary first temporal pattern in a region including a target position inside an object, using a magnetic field formation unit that generates a magnetic field;
generating one or more ultrasonic waves in which respective parameters of direction, frequency, amplitude, and phase are separately adjusted, by separately controlling one or more wave sources;
generating a force having a desired direction, intensity, and shape in accordance with the first temporal pattern so as to move a prescribed region of the object at the target position based on the force, by irradiating the target position with the one or more ultrasonic waves; and
generating an arbitrary electric potential based on the magnetic field in the prescribed region.

2. The electric potential generation method according to claim 1, comprising:
generating the force having a direction adjusted in accordance with a direction of the magnetic field generated based on the first temporal pattern at the target position so as to move the prescribed region in the direction of the force; and
generating an electric potential having an arbitrary second temporal pattern in the prescribed region.

3. The electric potential generation method according to claim 2, comprising:
generating the force so as to generate a velocity component in the same direction as the prescribed region for a time period that is long in comparison with a vibration period of the ultrasonic waves at the target position; and
generating a DC electric potential in the prescribed region for a prescribed time period.

4. The electric potential generation method according to claim 3, comprising:
generating the force in a direction substantially orthogonal to the direction of the magnetic field to move the prescribed region in the direction of the force; and
generating the DX electric potential in the prescribed region for an arbitrary time period.

5. The electric potential generation method according to claim 4, comprising:
generating the magnetic field that is a static magnetic field at the target position;
generating the force, which rotates in a planar direction substantially orthogonal to the direction of the magnetic field at the target position so as to generate torsional vibrations in the prescribed region in the direction of the force; and
generating the DC electric potential in the prescribed region for a prescribed time period.

6. The electric potential generation method according to claim 5, comprising:
outputting a plurality of the ultrasonic waves from different directions towards the target position based on a plurality of the wave sources disposed at different positions; and
generating a force rotating in a planar direction substantially orthogonal to the direction of the magnetic field, by combining the plurality of the ultrasonic waves at the target position.

7. The electric potential generation method according to claim 5, comprising:
making the ultrasonic waves generate vortex sound waves based on an acoustic lens; and
generating the force rotating in a planar direction substantially orthogonal to the direction of the magnetic field based on the vortex sound waves at the target position.

8. The electric potential generation method according to claim 5, comprising:
generating vortex sound waves, by combining the plurality of ultrasonic waves output from the plurality of wave sources disposed at different positions; and
generating the force rotating in a planar direction substantially orthogonal to the direction of the magnetic field based on the vortex sound waves at the target position.

9. The electric potential generation method according to claim 5, comprising:
generating the force so as to generate the torsional vibrations with a period causing the prescribed region to resonate; and
generating the DC electric potential in the prescribed region for the prescribed time period.

10. The electric potential generation method according to claim 6, comprising:
generating the force so as to generate the torsional vibrations with a period causing the prescribed region to resonate; and
generating the DC electric potential in the prescribed region for a prescribed time period.

11. The electric potential generation method according to claim 7, comprising:
generating the force so as to generate the torsional vibrations with a period causing the prescribed region to resonate; and
generating the DC electric potential in the prescribed region for a prescribed time period.

12. An electric potential generation apparatus comprising:
a magnetic field formation unit that generates a magnetic field having an intensity based on an arbitrary first temporal pattern in a region including a target position inside an object;
an ultrasonic wave output unit comprising one or more wave sources that generate ultrasonic waves; and
a control apparatus that controls the ultrasonic wave output unit and the magnetic field formation unit in a linked manner;
wherein the control apparatus
controls the magnetic field formation unit to generate a magnetic field in the region including the object, and
controls the ultrasonic wave output unit to generate one or more of the ultrasonic waves in which respective parameters of direction, frequency, amplitude, and phase are separately adjusted, and irradiate the target position with the ultrasonic waves so as to generate a force having a desired direction, intensity, and shape in accordance with the first temporal pattern, move a prescribed region of the object at the target position based on the force, and generate an arbitrary electric potential based on the magnetic field in the prescribed region.

13. A non-transitory computer-readable storage medium storing a program to be executed by a computer installed in a control apparatus that controls, in a linked manner, a magnetic field formation unit that generates a magnetic field having an intensity based on an arbitrary first temporal pattern in a region including a target position inside an object, and an ultrasonic wave output unit comprising one or more wave sources that generate ultrasonic waves,
wherein the program makes the computer;
control the magnetic field formation unit to generate a magnetic field in the region including the object; and
control the ultrasonic wave output unit to execute a process for generating, from the one or more wave sources, one or more of the ultrasonic waves in which respective parameters of direction, frequency, amplitude, and phase are separately adjusted, so as to generate a force having a desired direction, intensity, and shape in accordance with the first temporal pattern based on the one or more ultrasonic waves with which the target position has been irradiated, move a prescribed region of the object at the target position based on the force, and generate an arbitrary electric potential based on the magnetic field in the prescribed region.
